(19) United States
(12) Patent Application Publication
(10) Pub. No.: US 2008/0269889 A1
(43) Pub. Date: Oct. 30, 2008

(54) Haptic Junction Designs to Reduce Negative Dysphotopsia

(76) Inventor: Michael J. Simpson, Arlington, TX (US)

Correspondence Address: Jeffrey S. Schira, Esq.;Alcon Research, Ltd., Patent Dept., TB4-8, 6201 South Freeway, Fort Worth, TX, 76134, US

(21) Appl. No.: 11/741,818

(22) Filed: Apr. 30, 2007

(51) Int. Cl. A61F 2/16

(52) U.S. Cl. 623/6.46

(57) ABSTRACT

Methods and devices for inhibiting the dark shadow effect, known as dysphotopsia, perceived by some subjects having implanted intraocular lenses (IOLs) are presented. In one aspect, an IOL can include an optic and one or more fixation members for facilitating placement of the IOL. The fixation member can be adapted to have a portion that redirects light that is incident thereon in a manner which alleviates or prevents dysphotopsia. For example, the light that is incident on a fixation member can be directed to a retinal location intermediate to where imaging typically occurs on the retina and where a secondary image is formed. Various techniques for achieving these improvements are discussed, both in terms of the structures of improved IOLs, and methods for alleviating dysphotopsia.

### FIELD OF THE INVENTION

The present invention relates generally to intraocular lenses (IOLs), and particularly to IOLs that provide a patient with an image of a field of view without the perception of visual artifacts in the peripheral visual field.

### BACKGROUND OF THE INVENTION

The optical power of the eye is determined by the optical power of the cornea and that of the natural crystalline lens, with the lens providing about a third of the eye's total optical power. The process of aging as well as certain diseases, such as diabetes, can cause clouding of the natural lens, a condition commonly known as cataract, which can adversely affect a patient's vision.

Intraocular lenses (IOLs) are routinely employed to replace such a clouded natural lens. Although such IOLs can substantially restore the quality of a patient's vision, some IOL users report the perception of dark shadows, particularly in their temporal peripheral visual fields. This phenomenon is generally referred to as dysphotopsia.

Accordingly, there is a need for enhanced IOLs, and particularly for IOLs and methods that inhibit the perception of dark shadows in the peripheral visual field.

### SUMMARY OF THE INVENTION

The present invention is based, in part, on the discovery that the shadows perceived by IOL patients can be caused by a double imaging effect when light enters the eye at very large visual angles. More specifically, it has been discovered that in many conventional IOLs, most of the light entering the eye is focused by both the cornea and the IOL onto the retina, but some of the peripheral light misses the IOL and it is hence focused only by the cornea. This leads to the formation of a second peripheral image. Although this image can be valuable since it extends the peripheral visual field, in some IOL users it can result in the perception of a shadow-like phenomenon that can be distracting.

To reduce the potential complications of cataract surgery, designers of modern IOLs have sought to make the optical component (the “optic”) smaller (and preferably foldable) so that it can be inserted into the capsular bag with greater ease following the removal of the patient's natural crystalline lens. The reduced lens diameter, and foldable lens materials, are important factors in the success of modern IOL surgery, since they reduce the size of the corneal incision that is required. This in turn results in a reduction in corneal aberrations from the surgical incision, since often no suturing is required. The use of self-sealing incisions results in rapid rehabilitation and further reductions in induced aberrations. However, a consequence of the optic diameter choice is that the IOL optic may not always be large enough (or may be too far displaced from the iris) to receive all of the light entering the eye.

Moreover, the use of enhanced polymeric materials and other advances in IOL technology have led to a substantial reduction in capsular opacification, which has historically occurred after the implantation of an IOL in the eye, e.g., due to cell growth. Surgical techniques have also improved along with the lens designs, and biological material that previously affected light near the edge of an IOL, and in the region surrounding the IOL, no longer does so. These improvements have resulted in a better peripheral vision, as well as a better foveal vision, for the IOL users. It is interesting to note in this regard that the retina is a highly curved optical sensor, and hence can potentially provide better off-axis detection capabilities than comparable flat photosensors. In fact, though not widely appreciated, peripheral retinal sensors for visual angles greater than about 60 degrees are located in the anterior portion of the eye, and are generally oriented toward the rear of the eye. Though a peripheral image is not seen as sharply as a central (axial) image, peripheral vision can be very valuable. For example, peripheral vision can alert IOL users to the presence of an object in their field of view, in response to which they can turn to obtain a sharper image of the object. In some IOL users, however, the enhanced peripheral vision can lead to, or exacerbate, the perception of peripheral visual artifacts, e.g., in the form of shadows.

Dysphotopsia (e.g., negative dysphotopsia) is often observed by patients in only a portion of their field of vision because the nose, cheek and brow block most high angle peripheral light rays—except those entering the eye from the temporal direction. Moreover, because the IOL is typically designed to be affixed by haptics to the interior of the capsular bag, errors in fixation or any asymmetry in the bag itself can exacerbate the problem—especially if the misalignment causes more peripheral temporal light to bypass the IOL optic.

The present invention generally provides methods and devices (e.g., intraocular lenses (IOLs)), which can alleviate, and preferably eliminate, the perception of dark shadows that some IOL users report. Various embodiments of the present invention alleviate, and preferably prevent, dysphotopsia by adapting one or more optic fixation members of an IOL to direct at least some of the incident light onto a reduced intensity (shadow) region of the retina between an image formed by the IOL and a secondary peripheral image formed by light rays entering the eye that miss the IOL. Many such embodiments can help alleviate the perceived peripheral visual artifacts (e.g., shadows) without a substantial increase, if any, of the IOL's size. Accordingly, such IOLs can be deformed into a configuration suitable for delivery by minimally invasive methods.

In one aspect, an intraocular lens (IOL) is disclosed that includes an optic suitable for implantation in the eye of a subject, where the optic is adapted to form an image of a field of view upon the retina of the eye when the IOL is implanted in the subject's eye. The IOL can further have one or more fixation members coupled to the optic, which can be used to facilitate placement of the optic in the subject's eye. The fixation member(s) can include one or more portions that are adapted to receive some of the light rays entering the eye (e.g., entering the eye's pupil), and to direct those rays to the retina so as to inhibit (ameliorate and preferably prevent) the perception of peripheral visual artifacts (e.g., to inhibit dysphotopsia). Many such IOLs are deformable such that their delivery to a subject's eye is facilitated.

In a related aspect, in the above IOL, the portions of the fixation members that direct light to the retina to inhibit dysphotopsia comprise one or more light-directing elements. By way of example, such light-directing elements can inhibit dysphotopsia by directing at least some of the light incident thereon to a retinal location offset from a region of the retina in which the optic forms an image. Though the light-directing element(s) can potentially be located anywhere on a fixation member, in some embodiments the element is located in a region (e.g., a connection) between the optic and the fixation member body. For example, the light-directing elements can be disposed in a junction region connecting the fixation member to the optic. In many embodiments, such a junction region is positioned on the nasal side of the IOL in order to receive light rays (e.g., peripheral light rays) entering the eye from the temporal side. The light-directing elements can comprise any number of components, such as one or more Fresnel lenses and/or refractive surfaces and/or diffractive structures. By way of example, the light-directing elements can include one or more lenslets, and/or zonal regions, or any other suitable optical structures. Other examples include structures and/or coatings that diffuse light and/or scatter light in a manner to alleviate or prevent dysphotopsia. In some cases, the light-directing element(s) can have a designated focusing power, e.g., a focusing power less than that of the optic.

In another aspect, a deformable IOL includes an optic, one or more haptics coupled to the optic, and a junction region between the optic and at least one of the haptics, wherein the junction region can be adapted to direct light rays to one or more retinal locations offset from a retinal region in which the optic forms an image, so as to inhibit dysphotopsia. In some cases, the junction region can direct some of the light rays entering eye from the temporal side to the retina. In some cases, the junction region can provide a designated focusing power via, for example, a diffractive structure, a refractive structure, one or more lenslets, and/or a zonal region—such structures, however, can still be used to alleviate dysphotopsia without the designated focusing power. For instance, the focusing power can be less than that of the optic, or can be less than the optical power of the eye's cornea alone, or can be less than the combined optical power of the cornea and the optic, e.g., by a factor in a range of about 25% to about 75%). The junction region can also include a diffusive structure and/or a scattering structure, which can be adapted to direct light rays to a retinal location such as to prevent or alleviate dysphotopsia.

In another aspect, an IOL is disclosed, which includes an optic and one or more fixation members. At least one of the fixation members can include at least a portion (e.g., a diffractive structure) adapted to receive some light rays entering the eye and directing them to the retina so as to inhibit dysphotopsia. By way of example, the fixation member can be positioned on a nasal side of the eye, and can include a junction region for connecting it to the optic. The junction region can form the light-directing portion of the fixation member. Such an IOL can be deformable to facilitate its delivery to a subject's eye.

In another aspect, the invention provides an IOL, which includes an optic and one or more fixation members coupled to the optic. The IOL further includes a diffractive structure disposed on a surface of at least one of the fixation members. The diffractive structure can provide a focusing power that is less than the focusing power of the optic. For instance, the focusing power can be less than the optical power of the eye's cornea alone, or can be less than the combined optical power of the cornea and the optic, e.g., by a factor in a range of about 25% to about 75%). Alternatively, or in addition, one or more Fresnel lenses can be disposed on one or more fixation members to direct incident light. The focusing power of the Fresnel lens can be commensurate with that discussed with respect to diffractive structures.

In other aspects, methods of inhibiting the perception of visual artifacts (e.g., dysphotopsia) in a peripheral visual field of an IOL user are disclosed. Any of the IOLs described herein, which are effective for inhibiting dysphotopsia, can be implanted into the subject eye to help alleviate the perception of such visual artifacts.

In other aspects, methods are disclosed for inhibiting dysphotopsia in patients that have an IOL, wherein the IOL includes an optic and one or more fixation members coupled to the optic. The IOL can also include a junction region between the optic and a fixation member. Dysphotopsia can then be inhibited by altering the paths of at least some of the light rays that enter the eye's pupil and strike a portion of a fixation member (e.g., a junction region). For example, light rays that strike the fixation member can be redirected in a manner suitable to alleviate dysphotopsia, e.g., to one or more retinal locations in the eye offset from an image of a field of view formed on a retina by the IOL's optic. By way of example, such redirection can be accomplished by diffracting the light rays, refracting the light rays, or some combination of both.

### BRIEF DESCRIPTION OF THE DRAWINGS

Various features of embodiments of the present invention will be more readily understood from the following detailed description when read in conjunction with the appended drawings (not necessarily drawn to scale), in which:

FIG. 1 is a schematic cross-sectional top view of a left eyeball with an intraocular lens implanted therein.

FIG. 2A is a schematic anterior view of an intraocular lens, consistent with an embodiment of the invention.

FIG. 2B is a schematic perspective view of a fixation member of the intraocular lens depicted in FIG. 2A.

FIG. 2C is a schematic side view of the fixation member depicted in FIG. 2B.

FIG. 2D is a schematic side view of a portion of a fixation member suitable for use in some embodiments of the invention having a diffractive structure on a surface thereof.

FIG. 3 is a schematic cross-sectional top view of the left eyeball depicted in FIG. 1 with an intraocular lens having a light-directing aspect consistent with some embodiments of the invention.

FIG. 4A is a schematic anterior view of an intraocular lens with a junction region, consistent with an embodiment of the invention.

FIG. 4B is a schematic perspective view of a fixation member and a junction region of the intraocular lens depicted in FIG. 4A.

FIG. 4C is a schematic side view of the junction region depicted in FIG. 4B.

FIG. 5A is a schematic view of a folded intraocular lens consistent with an embodiment of the invention.

FIG. 5B is a schematic anterior view of the intraocular lens shown in FIG. 5A in an unfolded configuration.

FIG. 5C is a schematic anterior view of the unfolded intraocular lens shown in FIG. 5B rotated such that a junction region is proximal to the nasal side of the eye.

FIG. 5D is a schematic side view of the junction region between the lens and fixation member of the intraocular lens shown in FIG. 5A.

### DETAILED DESCRIPTION OF EMBODIMENTS OF THE INVENTION

The present invention generally provides ophthalmic methods and lenses (e.g., intraocular lenses (IOLs)), which can ameliorate, and preferably prevent, the perception of dark shadows that some IOL users report. Such an effect is known generally as negative dysphotopsia. Many embodiments are based on the discovery that such shadows can be caused by a double imaging effect when light enters the eye at very large visual angles, as described below.

The term “intraocular lens” and its abbreviation “IOL” are used herein interchangeably to describe devices that include one or more optics (e.g., lenses) that are implanted into the interior of the eye to either replace the eye's natural lens or to otherwise augment vision regardless of whether or not the natural lens is removed. Intracorneal lenses and phakic lenses are examples of lenses that may be implanted into the eye without removal of the natural lens.

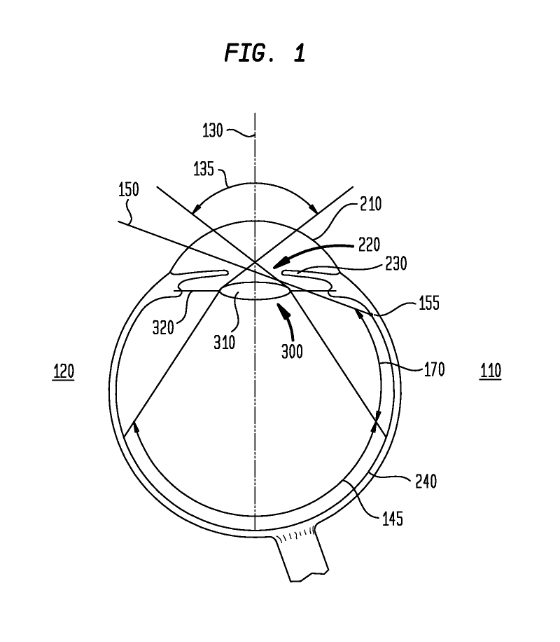

FIG. 1 presents a schematic cross-sectional top view of the left eyeball 100 of a subject having a conventional IOL 300 implanted therein. Light traveling from a field of view 135 passes through the cornea 210 and proceeds through the pupil 220 to impinge upon an optic 310 of the IOL 300. The combined optical power of the cornea and the optic focuses the light to form an image of the field of view on a region 145 of the retina 240. It has been discovered that in many conventional IOLs, which can be implanted in the posterior chamber of the eye, some of the light rays entering the eye at large visual angles (e.g., depicted by an exemplary light ray 150 in FIG. 1) miss the IOL's optic 310, passing through the space between the iris 230 and the optic 310, and are hence refracted only by the cornea to be incident on a portion of the retina 155 removed from the more central imaging region 145. Such light rays, herein termed “peripheral light rays,” typically enter from the temporal direction 120 and impinge upon the nasal side 110 of the retina as shown in FIG. 1. These peripheral light rays can form a secondary image or lighted region, with a reduced intensity region 170 linking the secondary image to the more central imaging region 145. The term “secondary image” as utilized herein is not strictly limited to a focused image on the retina, though peripheral light rays typically undergo focusing upon passage through the cornea. Indeed, such “imaging” can include any type of illumination of a retinal portion removed from the more central retinal region in which an image of field of view is formed by the focusing function of both the cornea and the IOL.

Though the presence of the secondary image can potentially aid in the peripheral visual perception of a subject, the separation of the two illuminated portions of the retina can result in the perception of a shadow-like phenomenon in a region between those images. It is hypothesized that this shadow-like perception is due to the presence of a reduced illumination region 170 on the retina between a primary image 145 and a secondary image 155. This phenomenon is known as negative dysphotopsia, and is typically perceived on the temporal side of the subject's field of view. The nose can block peripheral light rays from the nasal side, reducing the effect of the phenomenon in this direction. Dysphotopsia can also occur as a result of light reflection effects within an IOL's optic. Termed “positive dysphotopsia,” this effect can occur when the angular orientation of light entering the optic, combined with the index of refraction of the optic, results in total internal reflection of light rays within the optic, which subsequently exit the optic to form a secondary illuminated region on the retina.

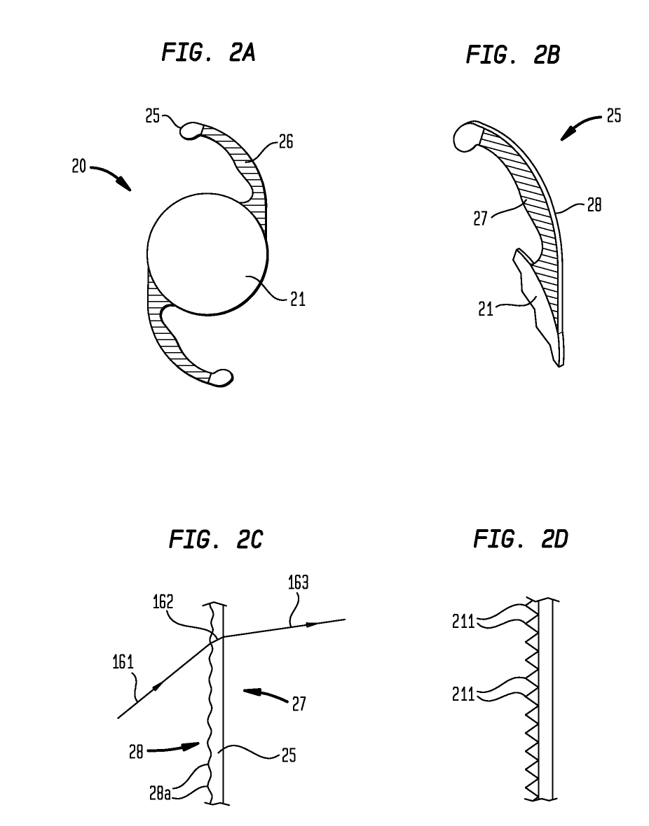

FIG. 2A presents an anterior view of an exemplary embodiment of an implantable IOL 20 according to the teachings of the invention, which is adapted to alleviate and preferably prevent dysphotopsia. The IOL 20 includes an optic 21 for forming an image of a field of view on the retina of a patient. One or more fixation members 25 can be coupled to the optic 21, which facilitate the placement of the optic in the eye. For the embodiment shown in FIG. 2A, the fixation members 25 are configured as two haptics (i.e., support structures coupled to a peripheral portion of the optic), which can couple to a structure of the eye (e.g., portion of the capsular bag, or a region between the root of the iris and the ciliary body) to provide a desired orientation of the optic. At least a portion 26 of a fixation member 25 can be adapted to receive some of the light rays entering the eye and to direct those rays to a region of the retina offset from where the image of a field of view formed by the cornea and the IOL is projected, so as to alleviate and preferably prevent dysphotopsia. By way of example, the portion 26 of the fixation member 25 can direct some of the light incident thereon onto the retinal reduced intensity region between an image formed by the IOL and a secondary peripheral image formed by rays that miss the IOL. It is understood that any number of fixation members can be configured to direct received light into a shadow region (e.g., only one haptic situated closer to a nasal side of the eye, or both haptics, can be so configured).

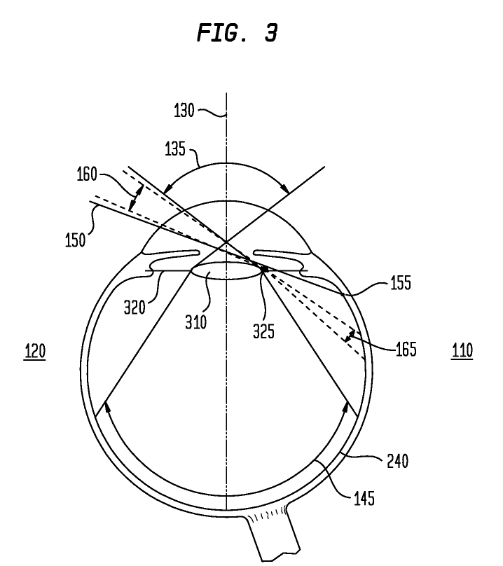

An example of how an IOL according to an embodiment of the present invention can alleviate dysphotopsia is provided herein with reference to FIG. 3, which schematically depicts the left eye of FIG. 1 in which an IOL is implanted. Peripheral light rays 150 can still be capable of forming a secondary image 155 on the retina 240, which can be potentially beneficial in enhancing peripheral vision. However, a reduced intensity (shadow) region between such a secondary image and a primary image generated by the IOL can lead to peripheral visual artifacts. To alleviate such visual artifacts, a portion 325 of a fixation member of the IOL can direct at least a portion of light rays 160 incident thereon to a portion of the shadow region 165. The light directed to illuminate the reduced intensity region 165 of the retina can be in various forms, such as a single light region or one or more discrete light regions (e.g., one or more “imaged” areas generated by using lenslets). Though this description is with reference to negative dysphotopsia, it is understood that an IOL can also alleviate or prevent positive dysphotopsia so long as the IOL is adapted to disrupt the reduced intensity shadow region between the typical imaging portion of the retina and the secondary image. Various embodiments of IOLs disclosed herein attempt to alleviate dysphotopsia utilizing a variety of structural features and techniques.

In some instances, light rays 160 that are incident on a portion 325 of a fixation member enter from a temporal side 120 of the eye as shown in FIG. 3. Configuring an IOL (e.g., an element 325 as depicted in FIG. 3) to redirect light rays that enter from a temporal side can reduce the angle of redirection required to illuminate a shadow region, which can be advantageous. It is also noted; however, that light rays incident on a portion 325 of the fixation member 320 need not be from a particular direction, so long as the portion 325 of the fixation member 320 is capable of directing the rays in an appropriate manner.

Optics utilized in a variety of the embodiments disclosed herein are preferably formed of a biocompatible material, such as soft acrylic, silicone, hydrogel, or other biocompatible polymeric materials having a requisite index of refraction for a particular application. For example, in some embodiments, the optic can be formed of a cross-linked copolymer of 2-phenylethyl acrylate and 2-phenylethyl methacrylate, which is commonly known as Acrysof®.

The term “fixation member” as utilized herein can refer to any structure that is coupled to an IOL's optic for positioning the IOL in a desired orientation upon implantation in a subject's eye, typically in a manner such that the optic acts as an effective optical aid to the subject. Similar to the optic 21, a fixation member 25 can also be formed of a suitable biocompatible material, such as polymethylmethacrylate (PMMA). While in some embodiments, a fixation member can be formed integrally with the optic, in other embodiments, the fixation member is formed separately and attached to the optic in a manner known in the art.

In some embodiments, a fixation member can include one or more light-directing elements, which can be used to direct light rays that are incident thereon in a desired direction, e.g., so as to inhibit dysphotopsia once the IOL is implanted in the eye. Light-directing elements can include any number of components assembled to guide light in a particular direction. Such elements include structures and/or coatings that can be formed either integrally with a fixation member, or manufactured separately and subsequently coupled to the fixation member. Some examples include zonal regions and/or lenslets that can be incorporated with a fixation member. Other examples include refractive and/or diffractive coatings or structures. Refractive coatings/structures can utilize any combination of material properties (e.g., interfaces between materials with different indices of refraction) and structural features which have a tendency to refract light in a particular manner. By way of example, diffractive coatings/structures can be embodied as a grating with a periodicity suitable for diffracting light in a given direction. Such diffractive elements can be tailored to diffract with one or more orders with particular efficiency. Another example of a light-directing element is the use of one or more Fresnel lenses to direct light rays. Further examples of light-directing elements include structures and/or coatings capable of diffusing light or scattering light in a manner to inhibit dysphotopsia, such as by directing the light into a reduced intensity region of the retina.

All these exemplary components of light-directing elements, among others (including those within the knowledge of one skilled in the art), can be used individually or in combination to provide light-directing capabilities in a fixation member. For instance, a light directing element can be formed from any combination of elements disposed on an anterior surface (i.e., the surface facing toward the cornea of the eye), a posterior surface, or both surfaces of a fixation member and/or a region of the IOL intermediate between the fixation member and the optic. In one example, it can be beneficial to place the light-directing element on an anterior surface, rather than a posterior surface, of the fixation member or a junction region to alleviate the potential risk of posterior capsule opacification (PCO)—though in other cases the light-directing element can be placed on the posterior surface or both surfaces. As well, the body of a fixation member and/or junction region, having anterior and posterior surfaces, can be adapted to also direct light in a particular manner. For example, the body can be a translucent body to cause diffusion of light passing therethrough. Such a body can be formed, in one example, by incorporating scattering centers in a biocompatible transparent polymeric material. Some examples of potential combinations are described herein with respect to FIGS. 2C, 2D, 4C, and 5C. Other combinations are also possible, such as configuring the fixation member to have an anterior surface that provides a refractive optical power.

In some embodiments, a light-directing element of the fixation member comprises a diffractive structure adapted to direct light incident thereon to a reduced intensity region between an image formed by the IOL and one formed by rays entering the eye that miss the IOL. Such a diffractive structure is schematically depicted in FIG. 2D, which is formed of a plurality of diffractive zones 211 separated from one another by a plurality of steps.

In use, a diffractive structure can direct at least some of the light rays incident thereon to a shadow region between a secondary peripheral image and an image formed by the IOL. In some implementations, the diffractive structure provides an optical power that is less than an optical power of the optic (e.g., by a factor in a range of about 25% to about 75%). As in many embodiments the diffractive structure receives off-axis peripheral light rays, it can be characterized as having an effective optical power for bending such peripheral rays (e.g., rays entering the eye at visual angles in a range of about 50 degrees to about 80 degrees) so that they would reach the reduced intensity region of the retina between an image formed by the optic and one formed by rays entering the eye that miss the IOL. For example, with respect to FIG. 3, the focal point of the diffracted light can occur beyond the retinal region 165. This can allow illuminating a larger portion of the reduced intensity region so as to alleviate dysphotopsia.

In other embodiments, the light-directing element of the fixation member comprises one or more Fresnel lenses which can be adapted to direct light incident thereon to a region of a retina for potentially alleviating dysphotopsia. For example, the structures 211 on the portion of the fixation member shown in FIG. 2D can be adapted to be one or more Fresnel lenses. The optical power of one or more of the Fresnel lenses can be less than the optical power of the cornea alone, or the combined optical power of the cornea and the IOL (e.g., such that the focal point is behind the retinal surface). For instance, the Fresnel lens can have a power in a range of about 25% to about 75% (e.g., about 50%) relative to the cornea or the combination of the cornea and the optic.

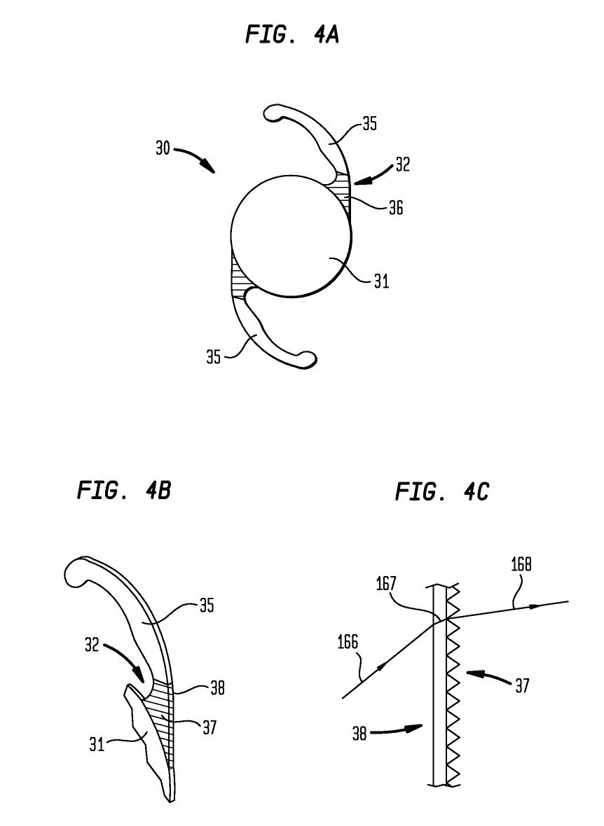

Returning to the specific IOL embodiment shown in FIG. 2A, a perspective view of one of the fixation members 25 of the IOL 20 is depicted in FIG. 2B. In the depicted embodiment, the shaded portion 26 of the fixation member 25, which directs light, can include an anterior surface 28 and a posterior surface 27 that are adapted to direct light that impinges on the portion 26. As shown in the side view of the fixation member 25 depicted in FIG. 2C, the anterior surface 28 can include a plurality of lenslets 28a, in the form of refractive surfaces that can refract light to a plurality of locations in the shadow region. By way of example, light ray 161 that strikes the anterior surface 28 is refracted to follow a new path 162 through the fixation member 25. It can then be further refracted by the posterior surface 27 to propagate to a location on the retina offset from the image formed by the optic so as to inhibit dysphotopsia. It is understood that the structures shown in FIGS. 2B and 2C are merely exemplary of how a light-directing element can be implemented in accord with embodiments discussed herein. Indeed, a light directing element can be embodied as any number of components, such as a transparent or translucent arm of a haptic having a posterior diffractive surface as discussed below with respect to FIG. 4C.

In some embodiments, a light-directing structure of the IOL that is adapted to receive and direct light rays to the retina so as to alleviate dysphotopsia can be located between the IOL's optic and its fixation member. For example, the structure can include a light-directing element that forms a part of, or an entirety of; a connecting junction between the fixation member and the optic. An exemplary embodiment is depicted in FIGS. 4A-4C. As shown in the anterior view of FIG. 4A, an IOL 30 includes an optic 31 and two fixation members embodied as haptics 35. A junction region 32 (also referred to as “junction” herein) connects a peripheral portion of the optic 31 and the haptic 35, and includes a light-directing element in the form of a diffractive structure 36 on an anterior surface 37 thereof as shown more clearly in FIG. 4B. The side view of the junction region 32 shown in FIG. 4C illustrates how the diffractive structure 36 redirects the light incident on the junction region. For example, light rays 166, incident on an anterior surface 38 are diffracted by the diffractive structure, on the junction's posterior surface 37, into a new direction, and pass through the junction body (which can be transparent or translucent to visible radiation) to be directed to the retina. In other embodiments, the diffractive structure can be disposed on the junction's anterior surface. Further, in some other embodiments, rather than utilizing the diffractive structure, the junction body can be translucent so as to cause sufficient diffusion of the light passing therethrough such that at least a portion of the light would reach the retinal reduced intensity (shadow) region.

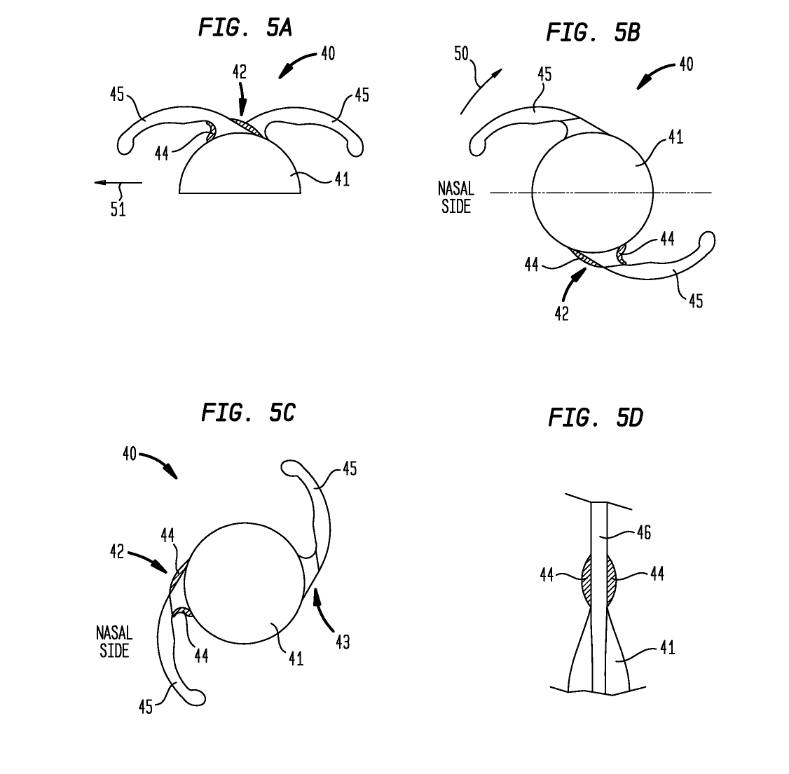

FIGS. 5A-5D schematically depict other exemplary features of IOLs according to some embodiments of the invention. For example, in many embodiments, the IOLs can be formed as deformable structures that can be delivered in a compact manner to an implantation site. As one example depicted in FIG. 5A, an IOL 40 can be folded in half for insertion in a direction 51 perpendicular to an incision. Accordingly, the size of the incision can be much smaller than that needed if the IOL was not folded. Upon delivery, such IOLs can unfold to an open configuration, as exemplified in FIG. 5B, and can be anchored by fixation members 45 in the eye. Typically, it can be desirable to make such IOLs as small as effectively possible to minimize the size of the incision needed to deliver the IOL. It is understood that other deformable configurations are also possible, such as deforming the IOL to fit in a tubular delivery structure.

Further, light-directing structures associated with a fixation member can be made advantageously small in some embodiments, which can be beneficial for limiting the size of the IOL. Further, it is advantageous in many embodiments to adapt a junction region between the haptic and the optic to redirect light to the reduced intensity region as such a junction can more readily receive peripheral light rays entering the eye.

In some embodiments, an IOL can include a junction having a portion (e.g., a light-directing element) that is oriented proximate to the nasal direction when the IOL is implanted in the eye. This can be advantageous since typically dysphotopsia is not associated with the other direction. As shown in FIGS. 5A and 5B, the IOL 40 can be oriented to enter an incision in the direction 51, the incision being substantially perpendicular to the insertion direction 51. After insertion, the IOL 40 can open into its operating configuration shown in FIG. 5B. The opened IOL can be rotated 50 into the orientation depicted in FIG. 5C such that the junction region 42 is proximal to the nasal side of the retina.

As dysphotopsia is generally perceived in the nasal retina, in many embodiments only the fixation member positioned on the nasal side is configured to direct some light into the shadow region, e.g., via one or more light-directing elements such as those discussed above. For example, as shown in FIGS. 5B and 5C, only one of the two haptics 45 depicted has a junction region 42 modified to direct light. Such embodiments can potentially reduce the size and expense of such IOLs.

The IOL depicted in FIGS. 5A-5D also exemplifies other potential features of a junction region that can direct light to the retina. For example, the junction region 42 can be expanded to include one or more extensions 44, each of which acts as a light directing element. As shown in the side view of the junction region 42 depicted in FIG. 5C, the extensions 44 can be a coating adjacent to the periphery of the optic 41. With the extensions acting as light-directing elements, such an embodiment can potentially redirect light rays without requiring transmission through a body portion 46.

In some embodiments, the IOL provides multiple focal powers. By way example, a diffractive structure can be disposed on an anterior surface (or a posterior surface or both surfaces) of the optic to provide the IOL with not only a far-focus (e.g., in a range of about −15 D to about 34 D) but also a near-focus optical power (e.g., in a range of about 1 D to about 4 D). In some cases, the optic's diffractive structure can be configured to include a plurality of diffractive zones that are separated from one another by a plurality of steps that exhibit a decreasing height as a function of increasing distance from the optical axis OA—though in other embodiments the step heights can be uniform. In other words, in this embodiment, the step heights at the boundaries of the diffractive zones are “apodized” so as to modify the fraction of optical energy diffracted into the near and far foci as a function of aperture size (e.g., as the aperture size increases, more of the light energy is diffracted into the far focus). By way of example, the step height at each zone boundary can be defined in accordance with the following relation:

$$Step height = \frac{λ}{a ( {n}_{2} - {n}_{1} )} {f}_{apodize} \qquad Equation ( 1 )$$

wherein

λ denotes a design wavelength (e.g., 550 nm),

α denotes a parameter that can be adjusted to control diffraction efficiency associated with various orders, e.g., a can be selected to be 1.9;

n₂ denotes the index of refraction of the optic,

n₁ denotes the refractive index of a medium in which the lens is placed, and

f_{apodize} represents a scaling function whose value decreases as a function of increasing radial distance from the intersection of the optical axis with the anterior surface of the lens. By way of example, the scaling function f_{apodize} can be defined by the following relation:

$${f}_{apodize} = 1 - {( \frac{{r}_{i}}{{r}_{out}} )}^{3} . \qquad Equation ( 2 )$$

wherein

rᵢ denotes the radial distance of the i^{th} zone,

rₒᵤₜ denotes the outer radius of the last bifocal diffractive zone. Other apodization scaling functions can also be employed, such as those disclosed in a co-pending patent application entitled “Apodized Aspheric Diffractive Lenses,” filed Dec. 1, 2004 and having a Ser. No. 11/000,770, which is herein incorporated by reference. In addition, further teachings regarding apodized diffractive lenses can be found in U.S. Pat. No. 5,699,142 entitled “Diffractive Multifocal Ophthalmic Lens,” which is herein incorporated by reference.

In this exemplary embodiment, the diffractive zones are in the form of annular regions, where the radial location of a zone boundary (rᵢ) is defined in accordance with the following relation:

rᵢ²=(2i+1)λf Equation (3)

wherein

i denotes the zone number (i=0 denotes the central zone),

rᵢ denotes the radial location of the i^{th} zone,

λ denotes the design wavelength, and

f denotes an add power.

IOLs according to various embodiments of the invention can be employed in methods of correcting vision. As discussed above, such IOLs advantageously inhibit the perception of visual artifacts in a peripheral visual field of the IOL user. For example, the IOLs can be employed to replace a clouded natural lens via cataract surgery. In cataract surgery, a clouded natural lens can be removed and replaced with an IOL. By way of example, an incision can be made in the cornea, e.g., via a diamond blade, to allow other instruments to enter the eye. Subsequently, the anterior lens capsule can be accessed via that incision to be cut in a circular fashion and removed from the eye. A probe can then be inserted through the corneal incision to break up the natural lens via ultrasound or other techniques. The lens fragments can be subsequently aspirated. An IOL, which can include an optic and at least one fixation member, can be implanted into a patient's eye (e.g., to replace the natural crystalline lens) to correct vision while inhibiting the perception of peripheral visual artifacts (e.g., dysphotopsia). For example, forceps can be employed to place the IOL in a folded state in the original lens capsule. Upon insertion, the IOL can unfold and its haptics can anchor it within the capsular bag.

In some cases, the IOL is implanted into the eye by utilizing an injector system rather than employing forceps insertion. For example, an injection handpiece having a nozzle adapted for insertion through a small incision into the eye can be used. The IOL can be pushed through the nozzle bore to be delivered to the capsular bag in a folded, twisted, or otherwise compressed state. The use of such an injector system can be advantageous as it allows implanting the IOL through a small incision into the eye, and further minimizes the handling of the IOL by the medical professional. By way of example, U.S. Pat. No. 7,156,854 entitled “Lens Delivery System,” which is herein incorporated by reference, discloses an IOL injector system. The IOLs according to the embodiments of the invention are preferably designed to inhibit dysphotopsia while ensuring that their shapes and sizes allow them to be inserted into the eye via injector systems through small incisions.

In some instances, dysphotopsia can be inhibited by altering a path of light that enters the pupil of the eye and strikes at least a portion of the fixation member. For example, the dysphotopsia can be inhibited by redirecting light rays that strike the fixation member. Such light rays can be redirected toward a retinal location offset from where an image of a field of view is provided by the IOL's optic. As discussed above, redirection of the light rays can be achieved by any one, or a combination of, refraction and diffraction of light rays that are incident on a fixation member.

IOLs that can be utilized with the exemplary method include any IOL suitable for practicing the method. Such IOLs include, but are not limited to, the IOLs that are taught or suggested in the present application. For example, the IOL can include a junction region between the optic and the fixation member, in which the junction region can include a portion adapted to alter the path of light rays that strike the portion. The portions of an IOL that can be used to direct light can include any of the light-directing elements disclosed herein.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments in any suitable combination. Accordingly, particular features with respect to described fixation members and light directing portions of such members (e.g., light-directing elements) can be chosen to construct alternative embodiments of the present invention. For example, the anterior and posterior surfaces shown in FIG. 2C can be used to replace the surfaces shown in the embodiments of FIGS. 4C and 5D, as well as adapted for use in any IOL disclosed herein. Such modifications and variations are intended to be included within the scope of the present invention. As well, one skilled in the art will appreciate further features and advantages of the invention based on the above-described embodiments. Accordingly, the invention is not to be limited by what has been particularly shown and described, except as indicated by the appended claims.

What is claimed is:

1. An intraocular lens (IOL), comprising:
a) an optic for implantation in an eye of a subject;
b) at least one fixation member coupled to the optic for facilitating placement of the optic in the eye; and
c) a light-directing element located between the fixation member and the optic, the light-directing element directing light rays incident thereon so as to inhibit perception of visual artifacts in the subject's visual field when the IOL is implanted in the subject's eye.

2. The IOL of claim 1, wherein the light-directing element is positioned on a nasal side of the eye.

3. The IOL of claim 1, wherein, upon implantation in the subject's eye, the optic is adapted to form an image of a field of view on a retina of the subject's eye, and the light-directing element is adapted to direct at least some light rays incident thereon to at least one retinal location offset from the image.

4. The IOL of claim 3, wherein the light-directing element comprises a posterior surface and an anterior surface, at least one of the posterior and anterior surfaces being adapted to direct light rays to the at least one retinal location offset from the image.

5. The IOL of claim 1, wherein the light-directing element comprises at least one Fresnel lens.

6. The IOL of claim 1, wherein the light-directing element comprises a diffractive structure.

7. The IOL of claim 1, wherein the light-directing element comprises a refractive structure.

8. The IOL of claim 1, wherein the light-directing element comprises at least one of a diffusive structure and a scattering structure.

9. The IOL of claim 1, wherein the light-directing element comprises at least one of a lenslet and a zonal region.

10. The IOL of claim 1, wherein the optic provides a first focusing power, and the light-directing element provides a second focusing power that is less than the first focusing power.

11. The IOL of claim 1, wherein the IOL is adapted to be deformable to facilitate delivery of the IOL to the subject's eye.

12. The IOL of claim 1, wherein the light-directing element forms at least a portion of a connecting junction between the optic and the at least one fixation member.

13. A method of inhibiting dysphotopsia in a patient having an implanted IOL, comprising:
implanting the IOL of claim 1 in the patient.

14. A deformable intraocular lens (IOL), comprising:
a) an optic for implantation in an eye of a subject, the optic adapted to form an image of a field of view on a retina of the subject;
b) at least one haptic coupled to the optic for facilitating placement of the IOL in the subject's eye; and
c) a junction region between the at least one haptic and the optic, wherein the junction region is adapted to direct light rays to at least one retinal location offset from the image so as to inhibit dysphotopsia.

15. The IOL of claim 14, wherein the junction region is adapted to direct some light rays entering from a temporal side of the eye to the at least one retinal location offset from the image.

16. The IOL of claim 14, wherein the optic provides a first focusing power, and the junction region comprises at least one portion providing a second focusing power less than the first focusing power.

17. The IOL of claim 14, wherein the junction region comprises at least one Fresnel lens adapted to direct light rays to the at least one retinal location offset from the image.

18. The IOL of claim 14, wherein the junction region comprises a diffractive structure adapted to direct light rays to the at least one retinal location offset from the image.

19. The IOL of claim 14, wherein the junction region comprises a refractive structure adapted to direct light rays to the at least one retinal location offset from the image.

20. The IOL of claim 14, wherein the junction region comprises at least one of a diffusive structure and a scattering structure adapted to direct light rays to the at least one retinal location offset from the image.

21. The IOL of claim 14, wherein the junction region comprises at least one of a lenslet and a zonal region adapted to direct light rays to the at least one retinal location offset from the image.

22. A method of inhibiting dysphotopsia in a patient having an implanted IOL, comprising the step of implanting the IOL of claim 14 in the patient.

23. A method of inhibiting dysphotopsia in a patient, comprising the step implanting into the patient an IOL with at least one fixation member adapted to redirect at least some light rays incident thereon to a retinal location between an image formed by the IOL and a secondary peripheral image formed by peripheral rays entering the eye that miss the IOL.

24. The method of claim 23, the step of redirecting light rays includes diffracting light rays that strike at least a portion of the at least one fixation member.

25. The method of claim 23, the step of redirecting light rays includes refracting light rays that strike at least a portion of the at least one fixation member.

26. An intraocular lens (IOL), comprising:
a) an optic adapted for placement in a patient's eye so as to form an image of a field of view; and
b) at least one fixation member coupled to the optic for facilitating placement thereof in the eye, the fixation member including at least a portion adapted to receive light rays entering the eye and directing such rays to a retina of the eye so as to inhibit dysphotopsia.

27. The IOL of claim 26, wherein the at least one fixation member is positioned on a nasal side of the eye.

28. The IOL of claim 26, wherein the portion of the at least one fixation member comprises a junction region connecting the at least one fixation member to the optic.

29. The IOL of claim 26, wherein the optic has a first focusing power, and the at least one fixation member has at least a portion with a second focusing power less than the first focusing power.

30. The IOL of claim 26, wherein the portion of the at least one fixation member comprises a diffractive structure for directing the light rays to at least one retinal location offset from the location at which the image is formed.

31. The IOL of claim 26, wherein the portion of the at least one fixation member comprises a Fresnel lens for directing the light rays to at least one retinal location offset from the location at which the image is formed.

32. The IOL of claim 26, wherein the IOL is adapted to be deformable to facilitate delivery of the IOL to the patient's eye.

33. A method of inhibiting dysphotopsia in a subject requiring the implantation of an IOL, comprising the step of implanting the IOL of claim 29 in the subject.

34. An intraocular lens (IOL), comprising:
a) an optic adapted for placement in a patient's eye so as to form an image of a field of view;
b) at least one fixation member coupled to the optic for orienting the optic in the patient's eye; and
c) a diffractive structure disposed on a surface of the fixation member.

35. The IOL of claim 34, wherein the optic provides a first focusing power, and the diffractive structure provides a second focusing power that is less than the first focusing power.

36. The IOL of claim 34, wherein the IOL is adapted to be deformable to facilitate delivery of the IOL to the subject's eye.

37. A method of inhibiting dysphotopsia in a subject requiring the implantation of an IOL, comprising the step of implanting the IOL of claim 34 in the subject.
